(19) United States
(12) Patent Application Publication
(10) Pub. No.: US 2004/0253323 A1
(43) Pub. Date: Dec. 16, 2004

(54) Ionic cancer therapy and methods for using same in the treatment of tumors and metastasis

(76) Inventor: Brian C. Giles, Montecito, CA (US)

Correspondence Address: Larry D. Johnson, Suite 203, 3558 Round Barn Blvd., Santa Rosa, CA, 95403, US

(21) Appl. No.: 10/867,115

(22) Filed: Jun. 14, 2004

Related U.S. Application Data
(60) Provisional application No. 60/477,678, filed on Jun. 11, 2003.

(51) Int. Cl. A61K 31/59; A61K 33/04; A61K 33/00

(52) U.S. Cl. 424/702; 424/722; 514/167; 514/557

(57) ABSTRACT

An expeditious method and formula for treating a wide variety of cancer neoplasm's administering anti-tumor agents for reduction or elimination of the path physiological process of cancer cell proliferation and the creation of an acidic micro-environment, resulting in stabilization, control, suppression, remission and elimination of metastatic seeding. The invention employs controlling mechanisms of intracellular and extra cellular ionic physiology through the administration of alkaline salts, optimally restoring localized and or systemic cellular ionic physiology and depriving cancerous cells of their ability to grow rapidly and simultaneously normalizing their local ionic environment, thereby inhibiting angiogenesis, reducing metastatic proliferation, stimulating immune responses and reducing pain.

### CROSS REFERENCE TO RELATED APPLICATIONS

The present application claims the benefit of the filing date of U.S. Provisional Patent Application, Ser. No. 60/477,678, filed 11 Jun. 2003.

### STATEMENT REGARDING FEDERALLY SPONSORED RESEARCH OR DEVELOPMENT

Not applicable.

### REFERENCE TO A MICROFICHE APPENDIX

Not applicable.

1. Technical Field

The present invention relates generally to the field of pharmacology and drugs for therapeutic elimination of metastases, cancerous tumors, and angiogenesis, and prevention of cancer.

2. Background Information and Discussion of Related Art

Cancer cells are different from normal healthy cells in several respects. One way in which virtually all cancer cells differ from normal healthy cells is that cancer cells derive a major proportion of their energy from glycolysis. Normal healthy cells utilize an oxidative metabolism in which only a small proportion of energy is derived from glycolysis. Cancerous neoplasm's require an alteration of energy production with transition from non-invasive premalignant to invasive malignant morphology, ranging from large benign tumors to necrotic cancers, including the acquisition of angiogenesis, increased glucose utilization (with increased lactic acid production) and typical tumor morphologies.

The increased lactic acid production of tumors causes the micro environment outside the tumor edge to become more acidic, leading to reduced pH. This decreased pH kills the normal tissue cells, which surround the tumor and which require a pH of 7.31 or higher to stay healthy and viable. As a consequence, the tumor is surrounded by necroticised normal cells. If insufficient alkalinizing agents are available to the healthy tissue cells surrounding the tumor, this promotes the extension (“invasion”) of the tumor into normal tissues. Increasing tissue permeability and the formation of new blood vessels (angiogenesis).

The values of pHe measured (the pH of the immediate environment of the tumor); vascularization, angiogenesis and surrounding tissue permeability correlate with invasiveness and metastasis. Low pHe makes tumor cell lines more metastatic.

The energy metabolism of tumor cells being acidic is uniquely different from normal healthy viable cells and provides a electro physical basis for selective destruction of stages of advancement and all varieties of neoplasm's that may lead to cancers as well as a wide variety of cancerous tumors.

Cancerous viability is dependent on an acidic micro-environment. This is due in part to their aberrant energy metabolism which produces lactic acid and carbonic acid and in part to incomplete vascularization, which causes insufficient oxygen supply (hypoxia).

The common denominator for virtually all tumors is a reduced pH at the tumor's edge. The tumors pHe (micro environment) ranges from 5.5 to 7.2 with an optimum growth rate occurring at a pH of 6.6 to 6.9.

Definitions: Acidity and alkalinity are measured by pH which is defined as the negative logarithm of the hydrogen ion activity: pH=−log (H). The parameter pHe is the pH on the exterior and pHi is the pH on the interior of the cell, as compared to systemic pH, which is the overall pH of the biological system.

The pH within tumor cells (pHi) is similar to (or even more alkaline than) the pH of normal tissue cells. The pH of the micro-environment of the tumor (pHe), however, is more acidic than that of normal tissues. It should be noted that the term “tumor micro-environment” refers to both the non-cellular area within the tumor and the area directly outside the tumorous tissue and does not pertain to the intracelluar compartment of the cancer cell itself.

Tumors tend to be both hypoxic and acidic. Chronically hypoxic tissues are going to be (i.e. are always) acidic, whereas transiently hypoxic tissues may be acidic. The more central part of the tumor is hypoxic, the exterior is transiently hypoxic.

Cancers exhibiting the lowest pHe values are more acidic and more aggressive and hostile to the surrounding normal healthy cells and more likely to be fatal to the patient. Metastasis is responsible for nearly 90% of cancer deaths. Low pHe promotes persistent antigenic and metastic signaling, metastatic spread of cancer and neovascularization (including angiogenesis, enhancing blood flow to the tumor mass). Low pHe decreases the efficacy of the immune response to cancer cells. An acidic hypoxic micro-environment causes genomic instability, and increased resistance to conventional cancer treatment procedures (e.g., drugs, radiation).

There are other important consequences of aberrant energy metabolism. As compared to healthy cells, cancer cells have a lower energy charge (ATP (ADP+Pi)). Additionally, all varieties of cancer cells typically have cellular distributions of ions that are different from normal healthy cells. Neoplastic and cancer cells usually contain excess internal sodium and grossly excess internal calcium, often with a deficiency in internal potassium. Cancer cells have ion fluxes across their membranes that are different from normal cells (e.g. increased H+ efflux). Cancer cells invariably have membrane electrical potentials (inside relative to outside) that are less electro-negative than normal cells. Aberrant ion concentrations such as high internal sodium or high internal calcium can induce apoptosis and or can modify the recognition of the cancer cell by the immune system.

Development of cancer involves a competition between the growth of neoplastic cells and their destruction by immunological processes. The genetic changes accompanying carcinogenesis have attracted great interest and much is known about the molecular mechanisms involved. Such changes are a prerequisite to the development of the malignant disease, but are not sufficient by themselves to overcome the immune defenses. Thus, cancer can be treated by therapies that potentiate the proper functioning of defenses such as immune response and apoptosis, so cancer propagation is shifted to promote cancer elimination.

The method and formula described in the invention have several related effects on development of the cancer micro-environment, both resulting from the same internal dynamics. First, the formula interferes with the hypoxic acidic energy metabolism of the cancer cells. This effect renders the cells less able to supply the energy required for the rapid proliferation typical of cancer cells resulting in a reduction or elimination in the viability zone of the cancer cells. Secondarily, the formula reduces acidification (both systemically and in the tumor micro- environment) and increases oxygenation, eliminating the adverse effects caused by acidic hypoxia.

The key to using aberrant energy metabolism as a way to specifically target the cancer cell's micro-environment is to administer a formula that has little or no toxicity to normal healthy viable cells and that increases the pH of the tumor micro-environment, thus causing the non-viability and apoptosis of tumor cells, leading to the elimination of tumors and metastases forcing the cancer cells to die or normalize. The formula reduces and eliminates the hypoxic acidification produced by the cancer cells so that the physiologic pHe range approaches 7.31 to 7.45. If pHe is close enough to this physiological optimum range. The anti-cancer activities of the immune system function in an optimal biochemical, ionic and electro-physiological environment. Malignant tumor survival requires neovascularization which preferentially occurs at reduced systemic pH and pHe. The induction of new blood vessels (neovascularisation angiogenesis) will be reduced or eliminated at more optimum alkaline pHe ranges. An acidic hypoxic micro-environment is a major factor in the induction of metastasis, propagation, seeding and adhesion. Therefore, the ionic formula of the invention will decrease or eliminate the risk of metastatic proliferation. A sufficient shift to the targeted alkaline levels will also mitigate the acidotic effects of tumor necrosis, thus reducing the need in some patients to surgically remove dead tumor tissue and reduce or eliminate tumor-generated pain. Such a therapy that take into account the consequences of low pHe and low systemic pH, that increases tumor pHe, without causing serious side effects is a significant improvement over the prior art.

Cesium and rubidium are alkali metals with chemical and physical characteristics similar to potassium. Potassium is the main internal cation of normal viable cells. Potassium ion currents are central to the ionic physiology of normal healthy viable healthy cells. Mammalian cells generally respond to hypoxic conditions that induce glycolytic energy metabolism with large potassium fluxes. Trans-membrane fluxes and cellular accumulation of cesium and rubidium ions are governed by similar cellular mechanisms as those which govern potassium movements however cesium and rubidium ions move at slower rates and accumulate to different degrees. This alters the ionic physiology of the cell, including inhibition of trans-membrane movement of potassium. Cesium and rubidium are effective for the control of potassium fluxes and linked hydrogen ion (H+) and other fluxes making them essential ions elements for therapeutic gain and prophylaxis suitable for all varieties of cancer by manipulating the pH of the tumor micro-environment and obtain a targeted shifting (raising) of the pHe and pHi of normal healthy cells to optimum or near optimum electro-physical range (outside cancers viability zone). Thus providing an electro-physical barrier to tumor growth, tumor invasion, metastasis seeding and neovascularization for all stages of malignancies as well as cancers resistant to conventional therapies, bypassing cancer cell's mechanisms of resistance against conventional anti-cancer drugs.

Prior Art Treatment Modalities: 1. Surgery: cancer therapies relies heavily on surgical intervention for the removal of the tumor load. There are high risks and stress associated with the surgery and post operative complications, high costs and high risks of life- threatening metastases. It is extremely difficult to be certain that the entire cancer is completely removed as residual cancer cells frequently survive and metastasize. Surgery produces pain (acidosis), reduction of which requires separate drugs that potentially contribute to chemical and psychological addiction.

2. Chemotherapy relies primarily on differential toxicity. Treatment in general is highly toxic to rapidly growing cells and often does not reach the tumor targeted edge. Some of these therapies often contribute to a further reduction of the systemic pH, thus further promoting acidic tumor micro-environment, compromising the patients' survival and recovery rate.

3. Radiation therapy's mode of action is by the causation of damage to rapidly growing cells. Radiation also causes permanent damage to non-cancerous normal healthy viable cells and contributes to the reduction of pH.

4. Stem cell therapy involves the use of both autologous and (matched) heterologous bone marrow-derived cells for replacing the immune cell population in various types of leukemia and lymphoma. This therapy requires extreme safety measures and is highly stressful for patients. In addition, it is costly and limited to a small number of malignant diseases.

5. Immunotherapy employ several forms of immune cells isolated from patients blood (e.g. dendritic cells, lymphokine activated killer cells) which, after in vitro stimulation with tumor antigens or immune modulators, are re introduced to the patient. A limitation of the immunotherapeutic approach is the limited number of tumor types that have successfully been treated (e.g. melanoma, kidney tumors), the expensive and complex procedure and the limited success rates.

6. Gene (Modulation) Therapy: this approach is still in its infancy. The major limitation is to achieve the required systemic specificity and efficacy.

These prior art therapies often adversely affect ionic function and interfere with the essential systemic pH, pHe and pHi balance and the critical enzymatic activities of normal cells and their ability to assimilate nutrients are compromised, substantially reducing there function. A superior approach to employ therapies that alter the tumor cells ability to transport H+ and other ionic species across its membrane. This provides a targeted shifting (increase) of the normal healthy cells and tissues pHe and pHi to a physiological optimum range outside the cancer cell's viability zone. This provides an electro-physical barrier to tolerate and resist pHe reduction and resistance to tumor formation and invasion, metastasis seeding and neovascularization. Resulting in tumor suppression and elimination, the current invention is separate from and superior to the above-referenced prior arts eliminating the short, and long term side effects so prevalent in the prior art.

The foregoing discussion reflects the current state of the art of which the present inventor is aware. Reference to, and discussion of, this information is intended to aid in discharging Applicant's acknowledged duty of candor in disclosing information that may be relevant to the examination of claims to the present invention. However, it is respectfully submitted that none of the above-indicated art discloses, teaches, suggests, shows, or otherwise renders obvious, either singly or when considered in combination, the invention described and claimed herein.

### BRIEF SUMMARY OF THE INVENTION

The present invention discloses a method and formula for administering a pharmaceutical composition to mammals and more specifically human patients suffering from cancer neoplasm's for eliminating and preventing the formation of a hostile cancer viability zone and tumor growing micro-environment. The invention is effective in treating a wide variety of cancers and for anti-metastasis and remission from cancerous tumors and will work on cancers with larger patient populations by inhibiting secretion of tumor proteases and the cancer propagation environment.

The method and formula may be administered as a short-term therapy, long term prophylactic therapy or maintenance therapy for life-long cancer remission. The invention employs a virtually nontoxic formula to electro-physically suppress the formation of the electro-physical source of the cancers viability zone on a cellular and ionic level. By administering Cesium and or Rubidium ions to neutralize and alkalinize the acidic hypoxic cancerous neoplasms. The common denominator for all cancerous neoplasms is that they require a reduced extra cellular pHe (microenvironment) which is more acidic than the intracellular pHi. The cancer cell's pHe microenvironment generally ranges from 5.5 to 7.2, with a growth rate peaking at a pHe of 6.8. All cancerous tumors have a narrow viability zone with a pHe ranging from 5.3 to 7.25. The Cesium and Rubidium ions have beneficial ionic characteristics for normal viable healthy cells that optimally function in a pHe range of 7.31 to 7.45 and a pHi range of 6.6 to 6.8. The Cesium and Rubidium ions raise the systemic pH and increase the alkaline ionic concentration in the tumor microenvironment, thus increasing tumor pHe and Oxidative Reduction Potential (ORP) to a physiologically optimum range for a controllable and predictable modulation of cancer apoptosis and or necrosis. The lactic and carbonic acid excreted by the cancer cells is dissociated at a physiological pHe range above 7.2. Such a response is instrumental in reduction and eliminating of tumor generated acidotic pain and systemic prevention and elimination of metastasis. The low extra cellular pHe is consequence of tumor growth and aggressively hostile to the growth and survival of normal viable cells and promotes tumor invasion and perfusion and further contributes tumorigenic transformation.

The ionic formula is administered to obtain sufficient ionic saturation of the tumor microenvironment for reducing or eliminating metastasis seeding and for promoting systemic pH resistance to cancer formation and suppresses and eliminates cancerous propagation and neovascularization in the tumor microenvironment. The formula administered to obtain ionic changes systemically and the pHe and pHi as well as the ionic chemistry of normal healthy viable cells, enhancing the ability of normal healthy viable cells and normal tissues surrounding the tumor edge to tolerate and resist decreased pHe and decreased pHi and to resist transport of H+ across its membranes, thus providing normal healthy cells and tissues an electro physical barrier to tumor growth invasion, eliminating the cancer viability zone (formation and propagation environment) and preventing the formation of the cancer-causing micro environmental conditions, which are a prerequisite for tissue invasion and metastatic seeding and the aberrant energy metabolism necessary for the viability of cancer formation and propagation. The formulas alkaline ions alter the tumor cells ability to transport H+ across its membrane, preventing the transition from non-invasive (pre-malignant) to invasive (malignant) morphology including the acquisition of angiogenesis, increased glucose utilization and increased acid production.

The therapeutic efficacy of the treatment in a particular patient can be assessed through observations relating to cancer ionic physiology. Clinical observation and diagnostic testing is used for adjustment of the dosages. Therapeutic dosages are adjusted in order to fall within the targeted pHi and pHe and systemic pH ranges; as an example saliva pH should range from 7.31 to 7.50, and only raise above 7.7, briefly the dose or dosages controls the degree, or rate, of efficacy, so that malignant tumor, suppression, and elimination occurs in a predictable and controllable manner, reducing the stress or mortality that often accompanies tumor necrosis. The invention discloses a non-addictive anti-cancer formula for readjusting ionic function to specific pH targets to obtain therapeutic gain for all stages of cancer including advanced staged cancers and suppresses cancer-induced tumor generated acidotic pain and inflammation. The pHe, pHi and systemic pH are restored to optimal function. Effects include those secondary to the inhibition of the large trans-membrane potassium movements resulting from hypoxic energy metabolism. The formula is administered to obtain shifts in pH, correction of excessive sodium accumulation, increase of membrane electrical potential and improvement of capacity of the sodium calcium exchange mechanisms. The formula promotes systemic hydration. Chronic dehydration is a major contributor to the cancer's physiological environment and the formation of acidic toxins.

The formula is generally administered to patients ranging from 15 to 30 consecutive days depending on dosages and rout of delivery to obtain tumor suppression and remission. The formula suppresses a wide variety of infectious microorganisms which often have acidotic energy metabolisms and thus are eradicated by raising the pH to more optimum physiological values. The formula has a high efficacy vs. toxicity ratio, enabling a large portion of the cancer treatment to be on an outpatient basis, resulting in substantial cost savings and reserving costly in-patient testing for patients with highly advanced and recalcitrant cancers.

The invention discloses a method for evaluating the saliva pH ranges to indicate a predisposition for the development of cancer, and provide a means for detecting cancer prior to the appearance of actual clinical symptoms and indicate the therapeutic dosages and corresponding efficacy for preventative measures and indicates the stage or progression of the cancer.

The method and formula can be used as an immuno modulatory therapy for a wide variety of cancers. The targeted goal of the invention is to manipulate pHi of normal healthy viable cells above 6.5 more preferably ranging from 6.4 to 6.8 for genetic repair and cancer prophylactic effects and stimulating the immune system to fight cancer cells.

The invention prevents recurrences of malignant tumors after the surgical treatment and increases the average life expectancy of patients with late stage cancers and avoids surgical treatment in a number of benign and malignant tumors.

The current invention is particularly effective in obtaining anti-metastasis activity and elimination of life threatening metastases. By administering an initial loading dosage, then administering therapeutic or maintenance dosages. By administering sufficient cesium and or rubidium ions and sufficient ionic saturation of the tumor microenvironment, eliminates metastasis electro physical environment. By shifting the pHe of healthy viable cells to an optimum range of 7.31 to 7.45 and by shifting (raising) the tumors micro environment (pHe) that generally ranges from 5.2 to 7.2.

The invention discloses a method and formula for gene repair and therapy. The disclosed method and formula that inhibits the transition from pre-malignant (non-invasive) to malignant (invasive) morphology including the acquisition of angiogenesis and increased glucose utilization (with increased lactic and carbonic acid production). The formula manipulates (raises) the pHi of healthy viable cells above the genetic pH mutation range to obtain gene stabilization and genetic repair locally and systemically. Low pH, hypoxia and nutrient deprivation can lead to mutations and result in genetic heterogeneity in tumors, thus cell lineages are selected with tolerance to tumor microenvironment and increased proliferation. For normal healthy viable cells the targeted ideal gene repair zone is a pHi above 6.41, preferably increasing to 6.6 to 6.8, thus extending the normal viability of cells, life span, and creating resistance against genetic damage and disease invasion.

The invention discloses a method and formula for improved chemotherapy. The formula lowers chemotherapy dosages, enhances cytostatic efficiency and enables healthy viable cells to reach a pHe close to the physiologically optimum range from 7.30 to 7.45. Low pH negatively affects the efficacy of chemotherapy and radiotherapy.

The formula enables normal tissues to tolerate and resist decreased pHe and decreased pHi, controls transport H+ across its membranes and provide an electro physical micro-environmental circulation barrier to tumor invasion.

The inventive formula eliminates tumor cell's ability to repair themselves after damage by chemotherapy or radiation, thus promoting enhancement of tumor necrosis after radiation- or chemotherapy. The formula reduces adverse symptoms of chemotherapy and radiotherapy by reducing the damaging effects of these procedures and the risk of DNA mutations caused by radiation and chemotherapeutic drugs, thus reducing the risk of causing further cancer in normal healthy cells.

The invention discloses a method and formula for immunotherapy and prophylaxis. The method and formula can be used as an immunomodulatory therapy of a wide variety of cancers.

The administration of the active salts in the formula with balanced electrolytes particularly including potassium and magnesium enhances the ability of immune cells including macrophages, natural killer cells and T-cells to obtain an optimal functional equilibrium, obtaining an improved immune surveillance for tumor cells. It is known that more acidic conditions (reduced pH) create a status of chronic immune stimulation and inflammation, which is evidenced by impaired functional immune balances. Restoration of the proper (slightly alkaline) pH not only strongly improves immune function, but also the interaction between the immune system and the psychoneuroendocrine system (e.g. hypothalamus-pituitary-adrenal axis) is positively influenced. Thus, this immunomodulatory treatment not only stimulates the immune reactivity towards cancer cells, but also can improve the mental health situation of cancer patients.

The invention discloses a method and formula for suppression of pathogens and disease resistance. The compromised immune system of cancer patients (as a consequence of inflammation, chemotherapy, radiotherapy) is often not capable of resisting microbial invasion. This apart from the impaired immune-surveillance regarding tumor cells. In addition, the expression of viral genetic material that may lead to cancer, is suppressed by eliminating the pathogen viability zone or pH environment.

The ionic formula enables normal healthy viable cells and tissues to act as a barrier to pathogen invasion which enhances the ability of cells to electro physically resist decreased pHe and pHi.

Administration of the formula increases the pH, stimulating immune response and obtains pathogen suppression, targeted shifting (rising) increasing the ionic concentration in the viable healthy normal cells pHe and pHi to physiological optimum and eliminates the reduction of pH, pHe, pHi, and mitochondria pHe and pHi. Additionally, extending the normal healthy viable cell's life span and enhances resistance against acid-mediated invasion of a wide variety of viruses and other opportunistic pathogens, such as bacteria, yeasts and fungi.

The invention discloses a method and formula for suppression of tumor generated pain Cancerous tumors generate lactic and carbonic acid and (H+) secretion lowering of the pH of the tumor microenvironment. This acid secretion is the cause of tumor-generated pain. The formula raises the pHe to a level that the lactic and carbonic acid disassociates at a pHe above 7.21 reducing or eliminating tumor generated pain. Thus the normal cells surrounding the tumor, including nerve and immune cells, function is restored including physiologically improve intracellular energy production and metabolism.

The invention discloses a method and formula for suppression and elimination of neovascularisation and angiogenesis. The formula is effective for repairing and normalizing neovascularization and preventing neovascularization. The invention method and formula obtains a targeted shifting (rising) of normal viable cell's pHe and pHi to physiological optimum values outside cancer's viability zone manipulates the neovascularization pH. The cesium and rubidium ions reduce or eliminate the lowering of pH pHe pHi and have angiogenesis inhibiting activity i.e. the formula inhibits the formation of new blood vessels. As an antiangiogenic agent, the ions will be effective in treating degenerative diseases which require a separate and new blood flow.

The invention discloses a method and formula for supporting surgical therapy. The formula suppresses infections during surgery and recovery and by promoting the healing cycle of normal surrounding cells, it enables a new method of wound care. In addition, residual tumor cells will encounter a more hostile environmental (more alkaline) pHe and, as a consequence, they are suppressed and eliminated.

The formula reduces or eliminates the recurrences of malignant tumors prior to, during, and after the surgical treatment and increases the average life expectancy of patients with late stage cancers. The anticancer therapy also prevents malignancy and metastasis and reduces shock during surgical treatment.

It is therefore an object of the present invention to provide a new and improved method whereby by manipulating the tumor's micro-environmental pHe range from between 5.2 and 7.2 to a physiologically optimum pHe range for normal healthy cells of 7.31 to 7.45, the tumor cells are brought out of their viability zone.

A further advantage of the invention is that it can cost-effectively be administered as a stand alone therapy or used as an effective adjunct in conjunction with a wide variety of conventional cancer therapies.

A further advantage of the invention is that it eliminates acidosis locally and systemically, thus rendering cancer cells non viable and lowering the potential risks of cancer metastasis by optimizing anti-cancer immune surveillance function by enhancing the capacity of healthy viable cells for disease invasion and resistance.

A further advantage of the invention is the possibility to co-administer alkaline ions with anti-metastatic treatment therapies useful in the (immuno) therapy of cancers, thus enhancing the ability of normal healthy viable cells and tissues to tolerate and resist acidic hypoxia invasions and decreased pHe and pHi and eliminating the tumors ability to transport essential ionic species (e.g. H+) across its membranes. Especially when used in combination with surgical treatment, prevent recurrent disease and increases the life expectancy of patients with late stage cancers.

Other novel features which are characteristic of the invention, as to organization and method of operation, together with further objects and advantages thereof will be better understood from the following description considered in connection with the accompanying drawing, in which preferred embodiments of the invention are illustrated by way of example. It is to be expressly understood, however, that the drawing is for illustration and description only and is not intended as a definition of the limits of the invention. The various features of novelty which characterize the invention are pointed out with particularity in the claims annexed to and forming part of this disclosure. The invention resides not in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is neither intended to define the invention of this application, which is measured by the claims, nor is it intended to be limiting as to the scope of the invention in any way.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### DETAILED DESCRIPTION OF THE INVENTION

The preferred embodiment discloses an ionic pH cancer therapy for inhibiting tumor metastatic activity for effective anti-vascularization and tumor suppression and remission for a wide variety of cancers as an example, but not limited to adenocarcinoma, adenocystoma, undifferentiated cancer, sarcoma, neurofibroxanthoma, fibroxanthoma, chondrosarcoma, osteosarcoma, lymphosarcoma, reticulosarcoma, carcinoma, carcinosacroma; adenoma prostate as well as mastopathy etc.

This invention discloses a method and formula of using alkaline salts, containing ions of cesium and or rubidium which meet certain ionic, electrochemical and electro-physiological cellular requirements, administered to target the cancer cells micro environment provide an effective dose of cesium and or rubidium ions, in combination with balanced electrolytes and supportive nutrients.

The therapeutic formula should not be administered with out sufficient quantities of balanced electrolytes including potassium and magnesium. Increasing the systemic pH in the cancer patient and eliminates acidic hypoxia, thus decreasing oxidative stress. Systemic alkalinization differentially raises tumor pHe due to the low buffering capacity of tumor interstitial fluid in the cancerous tumors' microenvironment. Cesium and rubidium ions taken up into the normal healthy viable cell tend to be released very slowly. When the acidity of a cancerous tumor's micro environment is shifted to a more electro-physiologically optimum pHe level above 7.31, the patient's metabolic and immune systems (including antibodies, macrophage cells, etc.) function more effectively and reduce and inhibit cancer cell replication. The pHe of the tumor microenvironment will be increased faster than the systemic pH. Such a response is instrumental in the prevention of cancer growth, invasiveness and anti metastasis and promotes tumor immunology response by the immune system.

If a patient's immune system is suppressed by a systemic or localized acidic hypoxic biological environment, either viral or bacterial-induced or tumor-generated, the therapy described herein will stimulate the immune response and function by obtaining a targeted increase of the systemic pH and pHe to physiologically optimum values and resist a wide variety of secondary infections.

METHOD OF MANUFACTURE/ PRINCIPAL ACTIVE INGREDIENTS: This invention utilizes salts containing cesium, rubidium ions, as a stand alone therapy, or as an effective adjunct in conjunction with a wide variety of conventional therapies administered in combination, in its manufacture, for example, but not limited to: any combination of salts containing ions of cesium and rubidium which disassociate and ionize may be employed in the formula as an example but not limited to: Arginate, Ascorbate, Caprylate, Cysteinate, Citrate, Fumarate, Methionine, Glutamate, Gluconate, Glycinate, Aspartate, Lysinate, Succate, Carnate, Lactate, Malate, Tartrate, Chloride, Sulfate, Phospate, Nitrate, Fluoride, Bromide, Iodide. There is variability in the ionization capability of the salts of cesium and rubidium with some ionizing more readily than others.

The toxicity of the salts of cesium and rubidium will depend on the salt combinations. As an example but not limited to the carbonate, chloride, citrate and sulfate salts are safer, the phosphate is relatively safe but may interact with calcium metabolism, while others have various levels of toxicity and should be used with caution. The organic species are preferred such as carbonate as the organic acids are readily metabolized.

The method and formula employs an alkaline salt solution formed by the following chemical formula: “MA”, where “MA” substantially dissociates in water solution to form “M+” and “A−”. “M” is the alkali metal moiety, which may be cesium and or rubidium. “A” is the anionic moiety, which may be any compatible nontoxic inorganic species such as but not limited to chloride, sulfate, carbonate or phosphate etc.; or it may be any compatible organic species such as lactate, citrate or acetate, etc.

In the event that it is necessary to combine the alkali metal moiety with an anionic moiety. For example, the hydroxide of the alkali metal can be combined with the acid form of the desired anion, thus: “MOH+HA=MA+H20”. In the case of acids that can dissociate more than one hydrogen ion, the final product may be partially protonated, for example, “MHCO2”, the bicarbonate salt, or “M2CO2”, the carbonate salt. The final formula can be designed by controlling the stoichiometry of the reaction, or by any known manufacturing process to obtain the required final pH and ionic concentrations. For example, citric acid can be used to neutralize a solution of cesium hydroxide until a pH near neutrality is obtained, or precise amounts of cesium hydroxide can be mixed with predetermined amounts of citric acid so that on dissolution a predetermined physiologic pH will be obtained. If tumor metabolism is monitored by lactate or lactate dehydrogenase (LDH) measurements, it may be preferable to avoid use of lactate to minimize background lactate or LDH signals. For orally administration the palatability will influence choice of anion(s) and the flavoring agent or agents selected.

The discrete dosage or ratios of cesium to rubidium salts employed will be governed by the therapies' efficacy and the physiological stress in the patient. In the event of physiological stress from high doses of one ion, a combination of various ions can be used to reduce the stress effects while retaining adequate therapeutic effectiveness. If salts of cesium or rubidium are not readily available then cesium rubidium in a pharmaceutically acceptable purity may be employed in the formula.

SECONDARY ACTIVE INGREDIENTS: These ingredients are chosen to complement or potentiate the action of the active ingredients. Some examples of potentiating ingredients are given to instruct the physician in the principals of their selection and are not intended to exclude other ingredients not mentioned. Potentiation of cesium and or rubidium ionic therapy can be accomplished by inclusion of ingredients that enhance the shift towards apoptosis induced by ionic physiology. Examples are compounds that stimulate calcium accumulation, such as calcium supplements with potassium and magnesium, preferably in a 2 to 1 ratio, vitamin D3, selenium salts, calcitonin, calcium ionospheres, etc., compounds that defeat the elimination of sodium from cancer cells such as Monessen or inhibitors of sodium and potassium exchange, compounds that alter pH regulating characteristics such as nigericin, amiloride and its derivatives, 4,4-diisothiocyanostilbene 2,2-disulfonic acid and bafilomycin. Further examples are compounds that decrease glucose utilization by tumor cells, such as ionidamine, and compounds that independently increase the activation of apoptosis.

Another class of ingredients which potentiate the activity of the primary active ingredients is those which stimulate or support the immune system and normal healthy viable cells, especially those which may be deficient as a secondary consequence of cancer, such as magnesium, zinc, vitamin B2 and B12. Ingredients that complement the salts of cesium and or rubidium therapy which may be useful in reducing cancer viability. These include the wide variety of chemotherapies and immunotherapy's that do not target ionic physiology. Because cancer development is a balance between the production and death of cancer cells, any additional ingredient that reduces the production or enhances cancer cell death can potentially be useful in the case of cancers recalcitrant to treatment with cesium and or rubidium therapy alone. Additional classes of ingredients which complement the ionic action of rubidium and or cesium salts therapy are those which minimize the toxic effects of tumor necrosis. These include systemic hydration, other alkalizing treatments, treatments that reduce the toxicity of solid tumor necrosis and are nutritional stimulation and dietary intervention or supplementation appropriate to the physiological stress associated with cancer or tumor necrosis. Additional classes of ingredients, which complement the action of rubidium and or cesium salts, are potassium, magnesium and other mineral supplements which compensate for potassium and other losses which may occur due to the mild diuretic effect of the therapy. Mineral supplements including trace minerals and ions are also used to obtain and maintain the desired pH range of bodily fluids and proper cellular metabolism.

Compounds intended to combat secondary infection such as antibiotics with antiviral, anti-bacterial, antifungal, and anti mold action may be included if appropriate. Chelation therapy may be included if appropriate to reduce or eliminate lead, mercury, cadmium, etc. Enzyme therapy may be included if appropriate.

PHYSICAL FORM: If a solidified crystalline formation of the salt or salts described herein is desired for purposes such as shipping or storage or oral administration, it can be manufactured by conventional methods containing buffered salt or salts. If preferred, the active ingredients may be orally administered without previous dissolution or they may be prepared as a pharmaceutical solution suitable for ingestion or injection using the aqueous carrier liquid. For example, solutions for injection should be prepared with a chemical composition that renders the solutions pH balanced and acceptable for injection. Typically, inject able solutions will be comprised of active ingredients in a sterile buffered saline solution isotonic to blood.

The water used in the aqueous solution may be from any suitable source. For manufacturing wide varieties of fluid concentrations as an example, but not limited to, the method of manufacture altering the electro-viscous characteristics by electro chemical activation (E.C.A.) restructuring the water molecules by processing such as, but not limited to, electrolytic treatment that provides sufficient voltage current intensity and duration (exposure) to external energy fields such as but not limited to electromagnetic, magnetic, radiation, sonic, etc. The active formula contains salts of cesium and or rubidium ions. As an example, lowering the surface tension from 73 dynes per cm2 to 55 to 68 dynes per cm2. Lowering the viscosity of water and restructuring the aqueous solution by increasing the hydrogen bond angle increasing from 101 degrees to 115 degrees more preferably 110 degrees to 114 degrees. This improves the patients systemic and cellular hydration, etc., producing an O.R.P. from −300 m.v. to −700 m.v. preferably −350 m.v. to −560 m.v., more preferred −400 m.v. to −500 m.v. For oral administration, the pH ranges from 8.5 to 9.7, preferably 8.6 to 9.3.

METHOD OF USE/MODES OF ADMINISTRATION: The alkaline salt solutions can be administered by any known method such as, but not limited to, periodic injections, intravenous infusion, rectal, vaginal, nasogastric, transdermal application, peritoneal, subcutaneous, sublingual, intramuscular, intrathecal, use of an implanted osmotic mini-pump or other slow-release methods or devices, etc. The injected solution is adjusted to be isotonic to blood and has a pH range of 7.36 to 7.44, and preferably 7.40.

For oral administration the aqueous formula is ingested simultaneously with ample alkaline processed water and a complex carbohydrate. Salts containing the active ions of cesium and or rubidium may take the form of gels, oils, bandages dressings, topical lotions, douche solutions, suppositories, colon irrigation solutions, or drop dispersions, encapsulated in liposome's, micro-particles, enteric coatings, micro capsules, transdermal patches, etc.

Discrete tablets may be made by compressing or molding, optionally with one or more secondary ingredients. Compressed tables may be prepared by compressing in a suitable method with the active ingredients in a free flowing form such as a powder or granuals, optionally mixed with a binder, lubricant, inert diluent, lubricating, surface active or dispersing agent. Molded tablets may be made by molding in a suitable method with a mixture of the powered compound moistened with an inert liquid diluent. The tablets may optionally be coated or scored and may be formulated so as to provide slow or controlled release of the active ingredient therein. Formulations of the present invention suitable for oral administration may be presented as discrete units such as capsule, cachets or tablets each containing a predetermined amount of the active ingredients; as a powder or granules; as a solution or a suspension in an aqueous liquid or a non-aqueous liquid; or as an oil in water liquid emulsion or a water in oil liquid emulsion. The active ingredient may also be presented as a bolus, electuary or paste. The formulations may be presented in unit-dose or multi-dose containers, for example sealed ampules and vials.

In general, the prescribed dosage required for therapeutic efficacy will be dependent on such factors as the patient's weight, age, nutritional background, gender, physical symptoms and condition, duration and frequency of administration, chosen route of administration, and the variety of cancer or cancers and its stage of advancement.

The oral lethal limit, LD-50, for cesium chloride is about 10 mEq kilogram in mice, there is no associated distress but some depression of activity at 5 mEq kilogram. The oral lethal limit for rubidium chloride is about 30 mEq kilogram in mice. The intravenous lethal limit is lower, 1.93 mEq kilogram in mice. The compounds of the invention may be administered orally or via injection at a dose from 1 to 100 mg kg per day preferably 3 to 50 mg kg per day. Doses above 3 mg kg per day may preferably be given in a series of smaller doses over a prolonged period, i.e. by infusion over several hours. The dose range for adult humans is generally ranges from 1500 mg to 7 g of the active salts per 24 hours and preferably ranging between 1200 mg to 3.5 g per 24 hours. Tablets or other forms of administration which are effective at such dosages or as a multiples of the same, for example, units containing 300 mg to 800 mg, usually ranging between 1000 mg to 3000 mg. As a general guide, the effective therapeutic dosage range for cesium is 0.1 to 5 mEq per kilogram daily for cancer therapy, 0.005 to 0.1 mEq per kilogram per 24 hrs. for cancer prevention, and 0.00001 to 0.01 mEq per kilogram per 24 hrs. for prevention of cancer related degenerative diseases. Doses over 1 mEq per kilogram should be used only for critical (life threatening) cases with careful monitoring for stress symptoms. Juvenile doses are generally about ½ of the adult range, depending on weight, etc. Cesium or rubidium salts should not presently be administered to pregnant or lactating females or infants, or patients with arrhythmias without further studies or appropriate medical intervention.

Injection doses for localized injection, such as directly into or near a primary tumor site utilize up to 300 mg of the active salts per kilogram of tumor mass per 24 hours. The degree of anti-tumor action required and the presence of any stress effects thus determines the route of administration dosage or amounts of cesium and or rubidium salts used. The formula and dose are adjusted (increased or decreased) as therapy progresses as indicated by the saliva pH urine pH and other indicators of therapy for ionic physiology. As an example, but not limited to, a patient suffering from cancer induced acute acidosis may be treated principally for that condition with cesium salts followed by a gradual introduction of rubidium salts or any effective combination to obtain a predictable degree of tumor necrosis. The discrete dose or dosages are determined to provide controllable rate of efficacy in cancer suppression to avoid stress due to excessive release of acidic toxins, for example due to large tumor volume necrosis. The method and formula are most effective if the diet is nutritionally stimulating and does not contribute to acidotic stress. As an example, dietary foods and beverages with a pH below 2.5 should be completely eliminated, or neutralized with calcium, etc.

Oral fluid consumption should be obtained primarily from E.C.A. processed water with a pH ranging from 7.5 to 9.3, and gradually increasing the fluid pH from 7.5 to 9.3. Foods with a low glycemic index are preferred and foods with a pH below 3 and beverages and foods whose low pH results from mineral acids such as phosphoric acid should be minimized. A neutral or slightly alkaline diet is maintained during the therapy.

EFFICACY: Regardless of the mode of administration, the patient's tumor or tumors should be monitored, as well as vital signs such as temperature and blood pressure. The patient's saliva, urine and blood pH should be monitored during the treatment and the dosage appropriately adjusted. The goal of dosage adjustment is to partially or wholly restore the optimum to pHe range from 7.35 to 7.45, preferably 7.4 during the therapy; both the pH of the fluid inside the tumor or tumors and the pH of blood emerging from a tumor should be monitored. As an example, magnetic resonance spectroscopy or other suitable methods including tissue sampling and analysis can be used to monitor pHe and pHi, and other indicating features of ionic therapy such as tumor sodium, potassium, magnesium and calcium levels and metabolites such as lactate. Further indications of dosage controlled efficacy are tumor stabilization, suppression or remission and the presence in blood or other body fluids of markers of tumor necrosis. A sufficient dosage that result in restoration of physiologic pHe and in a normal, as opposed to cancerous, ionic response to glycolytic metabolism are a short-term indication of the effectiveness of the therapy. Note that this therapy is effective for treating large volume solid tumors that must be confirmed by tumor regression. A lack of adequate response by the tumor pHe (micro environment) and other indicators suggests dehydration and or insufficient dosage.

STRESS: Excessive doses of rubidium and cesium salts can cause physiological stress, examples are mild diuretic effects, potassium depletion, low blood pressure or excessive alkalization. In cases where maximal efficacy is required during treatment, the upper dosage limit is set by the stress symptoms. The maximum dosage must be below the point at which perturbation of electrolyte balance causes damage. The pH measurements noted above for therapeutic efficacy will provide information useful for the physician in assessment of physiological stress. The patient is to be well hydrated before initiation of treatment. Dehydration should be monitored and promptly corrected if it occurs. Blood pH should not rise above 7.45, the targeted saliva pH range is from 7.3 to 7.5 only very briefly rise above 7.7, urine pH should not rise above 7.1 to 7.2 or below 5.0 or, only very briefly below 4.5. Additional symptoms of excessive pH rise are muscle aches or excessive numbness around the mouth. This indicates the need for temporary reduction in the dosage to avoid excessive alkalosis-induced stress and requires an increased dosage of electrolytes including potassium and magnesium until the symptoms are reduced to an acceptable therapeutic level. Doses should not exceed those that cause very slight sensations of numbness on the center of the upper lip and progresses around the mouth during therapy. Sensations of numbness indicate incipient effects on nerve tissue's ionic status. Blood potassium should not fall below tolerable levels. Blood pressure and serum creatine levels should be monitored.

### EXAMPLES

### Example 1

Use of cesium and rubidium for suppression and remission of the tumor microenvironment in tumor cancer therapy by oral administration. Amounts per 4 ounces bottle in electrolyzed aqueous solution containing cesium and or rubidium salts: Cesium citrate and or rubidium citrate, or any combination thereof, ranging from 500 mg per 24 hours to 5,000 mg per 24 hours, preferably 2,500 mg per 24 hours; potassium (preferably as phosphate, gluconate and or acetate) magnesium citrate 200-1500 mg; 500-2000 mg; and other nutrient supportive ingredients; calcium 2,500 mg-5000 mg; sodium chloride; iodine 100-400 mcg.; selenium (Selenomethionine) 50-200 mcg; vanadium (vanadyl sulfate) 2-10 mg; zinc gluconate 30-200 mg; vitamin D3 (cholecarciferol) 2,000 to 4,000 IU; vitamin A 2,000 to 5,000 IU; vitamin C buffered (L-ascorbic acid) 1,000 to 5,000 mg; malic acid 100-500 mg; coenzyme Q 10 (ubiquinone) 25-50 mg; vitamin B3 (methyl nicotinate) 20-30 mg; vitamin B6 25-100 mg; vitamin B12 (cyanocobalamin) 20-50 mg. The patient should be monitored for stress and efficacy as described herein, and dosages adjusted to obtain a tumor suppression and remission response with minimal physiological stress. Insufficient response, either initially or after a period of favorable response, indicates that complementary or potentiating ingredients should be considered.

### Example 2

Use of cesium and rubidium for anti-metastasis and anti-neovascularization therapy. Slow I.V. drip administration by injection into the bloodstream of a cancer patient using a sufficient quantity of salts of cesium and or rubidium. The I.V. solution and dosage per 24 hours generally ranges from 200 mg to 10 grams CsCl per liter, more preferably 1,200 mg per liter, and or from 200 mg to 10 grams RbCl per liter in a buffered saline solution made isotonic to blood. Any combination of CsCl and RbCl at the foregoing concentrations may also be utilized. As an example, solution administered by continuous intravenous drip (2×) per 24 hrs, generally ranging from 250 to 1000 cc as necessary. If the patient's physiological condition is life threatening such as comatose or terminal stage cancer, a higher dosage may be required, as an example, 1,000 cc two times per 24 hrs.

### Example 3

Oral capsules or tablets containing powdered salts of cesium and or rubidium. For administration to patients who are at high risk of reoccurrence of cancer after remission or as a prophylaxis dosage for long term remission. As an example, but not limited to: cesium citrate 400 mg; rubidium citrate 100 mg; potassium (preferably as phosphate gluconate and acetate) 150 to 1200 mg; calcium 2,500 mg; magnesium citrate 200-1,500 mg. The dosage of Cesium and or Rubidium salts administered in discrete tablets or capsules is preferably ingested with E.C.A. processed water administered with a carbohydrate. The active salt dosages generally range from 250 to 1000 mg. per 24 hours, more preferably 125 to 500 mg per 24 hours administered with orally administered nutrient support as an example, iodine 150-400 mcg.; selenium (Selenomethionine) 50-200 mcg; vanadyl sulfate 2-10 mg; zinc gluconate 50-200 mg; vitamin D3 (cholecarciferol) 2,000 to 4,000 IU; vitamin A 2,000 to 5,000 IU; buffered vitamin C-(L-ascorbic acid); 1,000 to 5,000 mg; malic acid 3-5 mg; co-enzyme Q 10 (ubiquinone) 25-50 mg; vitamin B3 (methyl nicotinate) 5-20 mg; vitamin B6 25-100 mg; vitamin B12 20-50 mcg. Monitoring the saliva pH and targeting the pH range is 7.3 to 7.5, only briefly up to 7.7. Monitoring the urine pH and not allowing it to rise above 7.1 to 7.2 or drop below 5.0, only very briefly below 4.5. The targeted urine pH is 7.0. Stress monitoring may be indicated if there are medical conditions that may be exacerbated by the pH selective therapy such as conditions relating to compromised or abnormal mineral absorption or low blood pressure.

### Example 4

Treatment of cancer using cesium and rubidium in combination with other anti-cancer agents. To lessen the risk of causing cancer in normal healthy cells when radiation therapy is used to treat cancer cells, or reduce the suppression of the immune response that often occur in conventional cancer therapies. The method for treating cancerous tumors includes a first treatment procedure using a conventional cancer treatment technique and a second treatment procedure which includes administering an effective amount of cesium and or rubidium. The first treatment procedure may be a treatment with one or more conventional cytotoxic chemical compounds. As examples, but not limited to the cytotoxic chemical compound may be a nitrosourea, cyclophosphamide, adriamycin, 5-fluorouracil, paclitaxel and its derivatives, cisplatin or other cancer treating agents. The cytotoxic chemical compound and the cesium and or rubidium may be administered by any suitable delivery route. The first treatment procedure may take place prior to the second treatment procedure, after the second treatment procedure, or the two treatment procedures may take place simultaneously. In one example, the first treatment procedure (e.g., discrete time intravenous dosage of cytotoxic chemical or chemicals) completed before the second treatment procedure with cesium and or rubidium administration begins. As an alternative, the first treatment procedure may be a conventional radiation treatment regimen. The method and formula for tumor remission and suppression of cancer may be used either preceding or subsequent to one or more conventional cancer therapies such as but not limited to radiation, cobalt, x-ray laetrile or cytotoxic treatment procedures. In this embodiment of the invention, the first method of treatment procedure is a radiation treatment, and the second treatment procedure is the administration of cesium and or rubidium salts. Radiation treatments can proceed on a schedule in combination with the administration of the formula to provide optimum results. Such scheduling of the treatment and procedures can be prescribed by the physician following routine medical evaluation. In addition to cesium and or rubidium salts formulations, the tumor also may be treated with one or more cytotoxic chemical compounds in a treatment. The use of salts of cesium and or rubidium in treatment modalities are separate and distinct from the use in radiation therapy of isotopes of cesium and or rubidium.

### Example 5

Another example of the invention is that cesium and rubidium salts can be incorporated into creams, lotions, solutions, ointments, gels, and other dermatologic formulations for the topical treatment of skin cancer for continuous topical administration one to two times daily, such as, but not limited to, squamous cell carcinoma, basal cell carcinoma, and cutaneous lymphoma, and for pre-malignant lesions such as actinic keratoses, and lesions of the skin such as psoriasis, seborrheic keratoses, and discoid lupus erythematosus. The physician should apply the dermatologic formulation to the affected area. The required duration of occlusion is variable, depending on the stage of advancement, such as, for example, the thickness of the cancerous lesions and the presence of thick scales, etc.

### Example 6

Administration, use of cesium and rubidium for gene-stabilizing and prophylactic therapy. Oral capsules or tablets powdered salts containing cesium and or rubidium ions for prophylactic administration or therapeutic maintenance of remission. This formulation is intended for use by patients who are at high genetic risk for the manifestation of cancer or as prophylaxis maintenance dosage. The dosages administered after cancer therapy are administered as one or more discrete tablets or capsules per 24 hrs administered with food. Dosage ranges from 15 to 30 mg, as an example, cesium citrate 10 mg.; rubidium citrate 10 mg.; potassium (preferably as phosphate, gluconate and or acetate) with supportive nutrients. As an example, dietary foods and beverages with pH below 2.5 should be completely eliminated, or neutralized pH with a suitable form of calcium, etc. Oral fluid uptake should be primarily derived from electrolyzed water with having a pH ranging from 7.5 to 9.3, preferably gradually increasing the pH of the fluids from 7.5 to 9.3. Foods with pH a ranging below 3 and beverages and foods whose low pH results from mineral acids such as phosphoric acid should be minimized. A neutral or slightly alkaline diet is maintained during the therapy. The patient should be monitored for stress and efficacy as described above, and the therapeutic doses adjusted to give a positive response with minimal stress. Failure to respond either initially or after a period of favorable response indicates that complementary or potentiating ingredients should be considered.

The use of novel methods employing the formula of the present invention have qualities that will be appreciated as this application encompasses broader and other aspects than recited in these examples.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting the scope of the invention, which is defined by the appended claims.

What is claimed as invention is:

1. A composition of matter comprising an aqueous alkali metal salt solution for use in the treatment of mammalian cancer, having the general formula:
MA_{(aq)}, wherein
MA dissociates in water to form M+ and A−;
M is an alkali metal selected from the group consisting of cesium and rubidium, and comprises cesium and rubidium either alone or in any combination thereof; and
A is an anion selected from the group consisting of chloride, sulfate, carbonate, phosphate, lactate, citrate, and acetate.

2. The composition of matter of claim 1 further including:
at least one substance to stimulate calcium accumulation, said substance selected from the group consisting of Vitamin D, selenium salts, calcitonin, and calcium ionophores;
at least one substance to reduce the elimination of sodium from cancer cells, said substance selected from the group consisting of monensin, and sodium/potassium exchange inhibitors;
at least one pH-modifying substance selected from the group consisting of nigericin, amiloride, 4,4′-diisothioscyanostilbene 2,2-disulfonic acid, and bifilomycin, in an amount sufficient to decrease acidity at the tumor site in the patient and systemic acidity in the patient; and
at least one substance to depress glucose utilization by tumor cells.

3. The composition of matter of claim 1 further including:
at least one substance in an amount sufficient to increase the activation of apoptosis in the patient;
at least one substance in an amount sufficient to stimulate the immune system, said substance selected from the group consisting of magnesium, zinc, Vitamin B2 and Vitamin B12;
at least one substance that complements cesium and/or rubidium therapy by unrelated means but which may be useful in reducing cancer viability, including compounds well known in the art and commonly used in chemotherapies that do not target ionic physiology; and
at least one substance in an amount sufficient to compensate for potassium loss due to any diuretic effect of the therapy, selected from the group consisting of potassium, anti-oxidants, and mineral supplements including trace minerals.

4. The composition of matter of claim 1 wherein said solution is suitable for oral administration twice daily in four ounce doses, said alkali metal salt is cesium citrate and/or rubidium citrate, or any combination thereof, in an amount ranging from 250 mg to 2,500 mg; and wherein said solution further includes 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandroststerone); 10 to 15 mg B3 methyl nicotinate; 12.5 to 50 mg B6; and 10 to 25 mcg B12.

5. The composition of matter of claim 1 wherein said alkali metal salt is cesium chloride and/or rubidium chloride, either alone or in any combination thereof, in an amount ranging from 200 mg to 10 grams alkali salt per liter of water, and said solution is buffered and isotonic to blood, said solution being suitable for administration by intravenous drip twice per 24 hours in an amount ranging from 250 to 2,000 cc, depending on patient needs.

6. The composition of matter of claim 5 further including 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandrosterone); 10 to 15 mg B3 methyl nicotinate; 12.5 to 50 mg B6; and 10 to 25 mcg B12.

7. The composition of matter of claim 1 wherein said alkali metal salt comprises 400 mg cesium citrate and 100 mg rubidium citrate; and further comprises 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandrosterone); 10 to 15 mg B3 methyl nicotinate; 12.5 to 50 mg B6; 10 to 25 mcg B12, and wherein the aqueous solution is processed for formulation into dry tablet or powdered form for oral administration twice daily to a cancer patient.

8. A method of treating mammalian cancer, comprising the steps of:
administering to a patient a therapeutically effective quantity of an aqueous alkali metal salt solution, wherein the alkaline salt has the general formula:
MA_{(aq)} and MA dissociates in water to form M+ and A−;
M is an alkali metal selected from the group consisting of cesium and rubidium, and comprises cesium and rubidium either alone or in any combination thereof; and
A is an anion selected from the group consisting of chloride, sulfate, carbonate, phosphate, lactate, citrate, and acetate.

9. The method of claim 8 wherein the aqueous alkali metal salt solution further includes at least one substance to stimulate calcium accumulation, said substance selected from the group consisting of Vitamin D, selenium salts, calcitonin, and calcium ionophores, and at least one substance to reduce the elimination of sodium from cancer cells, said substance selected from the group consisting of monensin, and sodium/potassium exchange inhibitors.

10. The method of claim 8 wherein the aqueous alkali metal salt solution further includes:
at least one pH-modifying substance selected from the group consisting of nigericin, amiloride, 4,4′-diisothioscyanostilbene 2,2-disulfonic acid, and bifilomycin, in an amount sufficient to decrease acidity at the tumor site in the patient and systemic acidity in the patient;
at least one substance to depress glucose utilization by tumor cells, and further includes at least one substance in an amount sufficient to increases the activation of apoptosis in the patient;
at least one substance in an amount sufficient to stimulate the immune system, said substance selected from the group consisting of magnesium, zinc, Vitamin B2 and VitaminB12; and at least one substance in an amount sufficient to compensate for potassium loss due to any diuretic effect of the therapy, said substance selected from the group consisting of potassium, anti-oxidants, and mineral supplements including trace minerals.

11. The method according to claim 8, wherein the alkali metal salt solution is orally administered.

12. The method according to claim 8, wherein the alkali metal salt solution is administered by injection.

13. The method according to claim 8, wherein the alkali metal salt solution is administered by introduction of said substance into a bodily cavity.

14. The method according to claim 8, wherein the alkaline salt solution is applied directly to cancerous neoplasms.

15. A method of treating mammalian cancer, comprising the steps of administering a therapeutically effective dose of an aqueous alkali metal salt solution, wherein the alkali metal salt is selected from the group consisting of cesium citrate, cesium chlroide, rubidium citrate, and rubidium chloride, either alone or in any combination thereof, in an amount ranging from 250 mg to 2,500 mg; and wherein said solution further includes 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandroststerone); 10 to 15 mg B3 methyl nicotinate; 12.5 to 50 mg B6; and 10 to 25 mcg B12.

16. The method of claim 15 wherein said alkali metal salt is cesium citrate and rubidium citrate, either alone or any combination thereof, in an amount ranging from 250 to 2,500 mg per four ounces of solution, administered orally twice daily.

17. The method of claim 15 wherein said alkali metal salt solution comprises aqueous cesium chloride and/or rubidium chloride, either alone or in any combination thereof, in an amount ranging from 200 mg to 10 grams alkali salt per liter of water, said solution buffered and isotonic to blood, said solution administered 250 to 2,000 cc per day by intravenous drip, depending on patient needs.

18. The method of claim 17 wherein the aqueous alkali metal solution further includes 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandrosterone); 10 to 15 mg B3 methyl nicotinate; 12.5 to 50 mg B6; and 10 to 25 mcg B12.

19. The method of claim 15 wherein said alkali metal salt comprises 400 mg cesium citrate and 100 mg rubidium citrate; wherein said solution further comprises 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandrosterone); 10 to 15 mg B3 methyl nicotinate; 12.5 to 50 mg B6; 10 to 25 mcg B12; said method including the further steps of formulating said solution into dry tablet or powdered capsule form for oral administration, said tablet or capsule being suitable for the long term treatment of mammalian cancer.

20. The method for treating mammalian cancer of claim 8 further including the step of monitoring pH and adjusting the therapy so that the systemic pH, the tumor pHe and the tumor pHi fall within a predetermined range.
